(19) United States
(12) Patent Application Publication
(10) Pub. No.: US 2013/0025004 A1
(43) Pub. Date: Jan. 24, 2013

(54) GRAPEVINE PLANT denominated 'Iniagrape-one'

(71) Applicant: Carolina Uquillas, Santiago (CL)

(72) Inventor: Carolina Uquillas, Santiago (CL)

(73) Assignee: INSTITUTO DE INVESTIGACIONES AGROPECUARIAS, Santiago (CL)

(21) Appl. No.: 13/554,669

(22) Filed: Jul. 20, 2012

(30) Foreign Application Priority Data
Jul. 22, 2011 (CL) 1148

(51) Int. Cl. A01H 5/00

(52) U.S. Cl. 800/298

(57) ABSTRACT

A new and distinct variety of grapevine denominated ‘Iniagrape-one’ which is characterized by its midseason ripening. Its fruit is attractive blue-black, with seed rudiments, broad ellipsoid-narrow ellipsoid fruit shape, difficult berry separation from pedicel, firm texture with very good particular flavor because its acidity and sugars-acid ratio. The clusters are large in size, conical and medium density. Good productive capacity and keeping and shipping quality.

### BACKGROUND AND SUMMARY OF THE INVENTION

The present invention relates to the discovery and asexual propagation of a new variety of grapevine, Vitis vinifera L. denominated Iniagrape-one. This new cultivar resulted from controlled hybridization between Flame Seedless (♀) and Black Seedless (♂) performed in 1998 at La Platina Experiment Research Center in Santiago Chile (latitude 33° 34,32 S, longitude 70° 37,41 S, altitude 631 meters above sea level), corroborated by molecular markers techniques. Plants were produced from maternal parent Flame Seedless using embryo rescue procedures (Emershad and Ramming, Amer. J. Bot. (1984), 71:873-877; Emershad et al., Amer. J. Bot. (1989), 76:397-402; Ramming, HortScience (2008) 43(3):945-946). The original vine was planted in 2000 at La Platina Experiment Research Center in Santiago Chile, and selected in 2004.

‘Iniagrape-one” is vigorous and produces a strong vine when grafted on Freedom or Harmony rootstocks. Cane or spur pruning may be used because of its high bud fruitfulness along the shoots. In the central area of Chile (Aconcagua Valley 32° 52 S, 70° 38 W)), budbreak occurs around September 22^{nd}, and flowering around November 15^{th}.

The fruit is blue-black, ellipsoid shape, with large berries, about 8 gr. when is sprayed with gibberellic acid, containing 2 to 3 rudimentary soft seed traces per berry. Occasionally, depending on location and the season, some few berries contain 1 seed.

This behavior is similar to Autumn Royal cultivar. The clusters are large in size, about 600-800 gr., conical and medium density. The fruit has a good export market potential for having good keeping and shipping quality.

‘Iniagrape-one’ is harvested in the middle of February in the central area of Chile (Aconcagua Valley 32° 52 S, 70° 38 W)), one week after Black Seedless and Midnight Beauty and 3-4 before Autumn Royal cultivar. ‘Iniagrape-one” differs from those varieties for its complete uniform blue black color without the need for any hormonal spray. It has a distinct particular good flavor gives by ration sugar acidity at harvest time (19-21° brix and 0.9-1% acidity). It also differs from Black Seedless for having better keeping and shipping quality. The new variety has no problem of shattering during harvest or post-harvest due to a difficult separation of the berry pedicel. For the species Midnight Beauty separation of the berry pedicel has been classified as medium, and in Autumn Royal the union between the rachis and the pedicel is fragile, and berries can be separated from the rachis during harvesting. Unlike Autumn Royal, ‘Iniagrape-one’ has no tendency to crack.

The new ‘Iniagrape-one’ variety has shown to maintain its distinguishing characteristics through successive asexual propagation by, for example, cuttings.

### BRIEF DESCRIPTION OF THE FIGURES

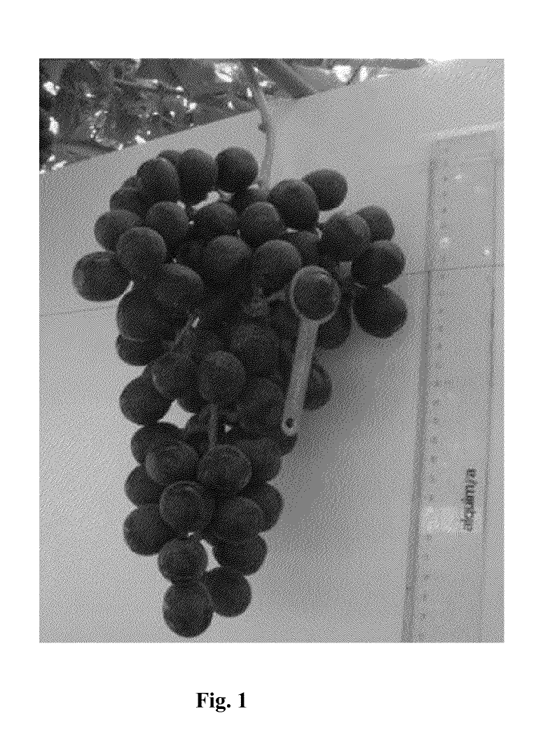

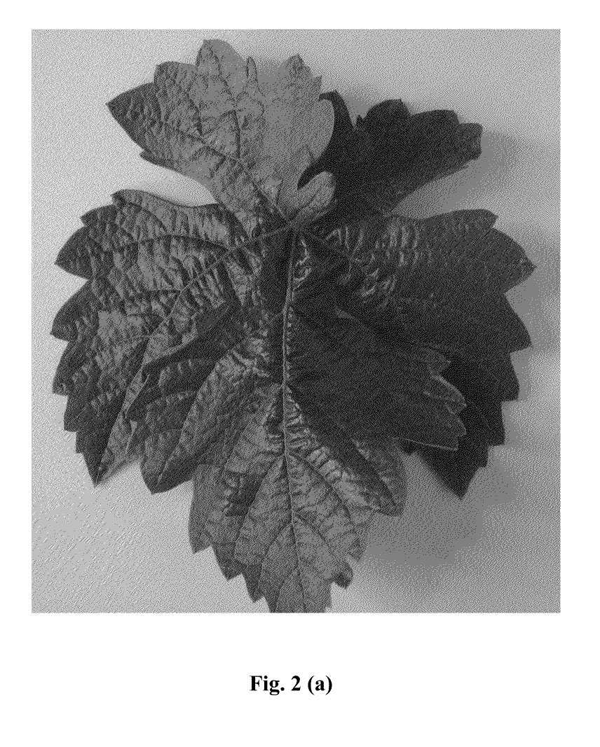

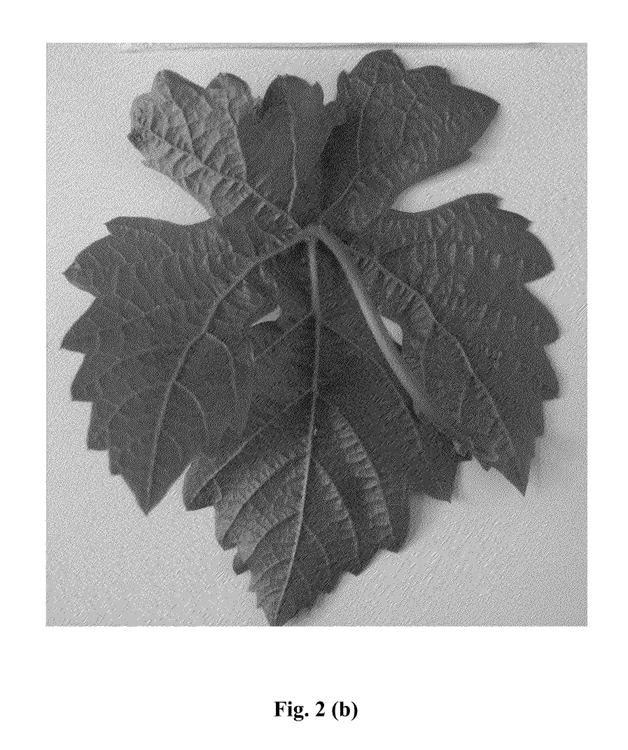

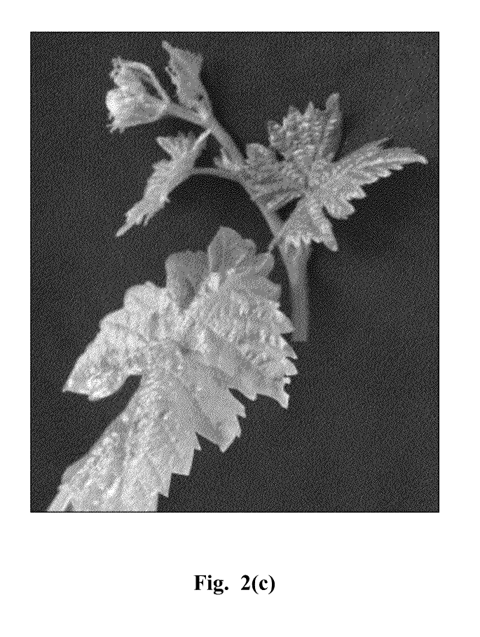

The drawings of the grapevine of the present invention are color photographs showing in FIG. 1 a typical cluster of ‘Iniagrape-one’. FIG. 2(a) corresponds to the upper surface of a mature leaf and FIG. 2(b) correspond to the lower surface of a mature leaf. FIG. 2(c) corresponds to a shoot with young leaves. Description applies to vines grafted on Freedom.

### DETAILED BOTANICAL DESCRIPTION

The following botanical description represents a three season evaluation observed in an approximately four to six year old grapevine. In all sites, vines are cane pruned on an overhead trellis system (grape arbor) and the descriptive data are from commercial handled clusters.

### Leaves

Young Leaves:
Color of upper surface of first 4 distal unfolded leaves.—Yellow with bronze spots.
Color of lower surface of first 4 distal unfolded leaves.—Green. Density of prostrate hairs between veins at lower surface of 4th distal unfolded leaf-Absent.
Density of erect hairs between veins at lower surface of 4th distal unfolded leaf.—Absent.
Density of prostrate hairs on veins at lower surface of 4th distal unfolded leaf.—Sparse.
Density of erect hairs between veins at lower surface of 4th distal unfolded leaf.—Absent.

Mature Leaves:
Average length.—About 18.5 cm.
Average width.—About 11.5 cm.
Size of blade.—Medium.
Shape of blade.—Pentagonal.
Number of lober.—5.
Anthocyanin coloration of main veins on the upper side of the blade.—Absent.
Mature leaf profile.—Undulate.
Blistering surface of blade upper surface.—Weak.
Shape of teeth.—Mixture of both sides straight and both sides convex.
Length of teeth.—Medium.
Ratio length/width of teeth.—Medium.
General shape of petiole sinus.—Lobes slightly overlapped
Tooth at petiole sinus.—Absent.
Petiole sinus limited by veins.—Absent.
Shape of upper lateral sinus.—Lobes slightly overlapped.
Depth of upper lateral sinus.—Deep.
Prostrate hairs between veins on lower surface of blade.—Absent or very sparse.
Erect hairs between veins on lower surface of blade.—Absent.
Density of prostrate hairs on main veins on lower surface of blade.—Very sparse.
Density of erect hairs on main veins on lower surface of blade.—Absent or very sparse.
Prostrate hairs on main veins on upper surface of blade.—Absent.

### Flowers

### General:

Flower sex.—Perfect.
Position of first flowering node.—From 3rd node.
Number of inflorescences per shoot.—About 1 to 2.
Date of full bloom.—Around November 15^{th}, Central valley of Chile.
Duration of bloom period.—On average, about 7 days.
Time of bloom.—Medium, around, November 8^{th}, Central valley of Chile.
Calyptra color.—Green.
Ovary color.—Green.
Stamens.—Erect.
Pistil.—Well developed.

### Fruit

### General:

Ripening period.—Midseason. About one week after of “Black Seedless” and “Midnight Beauty” and three to four weeks ahead “Autumn Royal” in the growing area of Aconcagua valley (Los Andes), Chile.
Use.—Fresh market.
Keeping quality.—Good.
Shipping quality.—Good.
Date of first harvest.—Feb. 19, 2008.
Solids-sugar.—Refractometer test about 19-21° Brix.
Acid.—Acid about 0.9-1%.
Juice pH.—About 3.5.
Sugar/acid ratio.—About 20.
Tendency to crack.—None.
Sensitivity to sunburn.—None.

### Cluster:

Bunch size (peduncle excluded).—Large.
Bunch length (peduncle excluded).—About 19-20 cm.
Bunch width.—About 15-17 cm.
Bunch weight.—Between 600-800 gr.
Bunch density.—Medium.
Number of berries.—Between 90-120.
Form.—Conical.

### Peduncle:

Length of peduncle.—About 4-5 cm.
Lignification of peduncle.—Medium.
Color.—Green.

### Berry:

Size.—Large.
Uniformity of size.—Medium.
Berry weight.—About 8 gr.
Shape.—Broad ellipsoid and narrow ellipsoid.
Presence of seeds.—Rudimentary. Berries containing 2 to 3 rudimentary soft seed traces. Occasionally, depending on location and the season, some few berries contain 1 seed.
Cross section.—Circular.
Dimensions.—Longitudinal axis about 24 mm; horizontal axis about 22 mm
Skin color (without bloom).—Blue black.
Flesh color.—Weak.
Juiciness offlesh.—Juicy.
Berry firmness.—Firm.
Particular flavor.—Other than Muscat, foxy or herbaceous.
Bloom (cuticular wax).—Medium.
Pedicel length.—About 10 mm
Berry separation from pedicel.—Difficult.

### Skin:

Thickness.—Thick, about 0.5 mm
Texture.—Smooth.
Reticulation.—Absent.
Roughness.—Absent.

### Seed:

According to observations of three productive seasons and three different locations, the variety was phenotypically classified as rudimentary. The berries contain 2 to 3 red rudimentary soft seed trace per berry. Occasionally, depending on location and the season, some few berries contain 1 seed.

### Vine

### General:

Size.—Large.
Vigor.—Vigorous.
Density offoliage.—Dense.
Productivity.—Very productive, about 24 to 27 Kg/vine.
Root stock.—Freedom.

### Canes

### Young Shoot:

Form of tip.—Fully open.
Intensity of anthocyanin coloration of tip.—Absent or very weak.
Density of prostrate hairs on tip.—Medium.
Density of erect hairs on tip.—Absent or very sparse.

### Shoot:

Attitude during flowering on shoot which are not tied.—Semi-erect.
Color of dorsal side of internodes.—Green and red.
Color of ventral side of internodes.—Green and red.
Color of dorsal side of node.—Green and red.
Color of ventral side of nodes.—Green and red.
Density of erect hairs on nodes.—Absent or very sparse.
Erect hairs on internode.—Absent or very sparse.
Density of prostrate hairs on nodes.—Absent or very sparse.
Density ofprostrate hairs internodes.—Absent or very sparse.

### Woody Shoot:

Internode length.—Short or medium, about 5.5 to 13.0 cm.
Cross section.—Circular.
Surface texture.—Smooth.
Main color.—Yellowish brown.

### Buds:

Cane bud fruitfulness.—Basal most fruitful.
Time of bud burst.—September 22^{nd}, Central area.

### Tendrils:

Length of tendrils.—Medium, about 19 to 21 cm.

having thus described and illustrated the new variety of grapevine, I claim:

1. A new and distinct variety of grapevine as herein illustrated and described.
